(19) United States
(12) Patent Application Publication
(10) Pub. No.: US 2011/0060201 A1
(43) Pub. Date: Mar. 10, 2011

(54) Integrated Pulse Oximeter-Pulse Flowmeter

(76) Inventors: Lloyd A. Marks, Westfield, NJ (US); Michael Smith, Oradell, NJ (US)

(21) Appl. No.: 12/555,400

(22) Filed: Sep. 8, 2009

(51) Int. Cl. A61B 5/1455; A61B 5/02

(52) U.S. Cl. 600/324; 600/506

(57) ABSTRACT

This device uses electrical impedance and signal processing to measure the small change in volume (“Pulse Volume”) of a limb-segment that occurs with each heartbeat. “Pulsatile Flow” is defined as the pulse volume multiplied by the heart rate and is an index of tissue perfusion. This information can be combined with measured blood oxygen saturation to determine how much oxygen is reaching the tissues. The combination of knowing oxygen saturation and tissue perfusion is far more useful than knowing just one or the other of these parameters.

### FIELD OF THE INVENTION

The present invention is directed to a pulse oximeter and more particularly to an integrated pulse oximeter and pulse flowmeter for improved indication of how well the body tissues are being oxygenated and/or perfused

### DESCRIPTION OF RELATED ART

Hemoglobin, the substance in red blood cells that carries oxygen, exists in two forms. Oxyhemoglobin, the hemoglobin which is carrying oxygen, is bright red. Deoxyhemoglobin, hemoglobin without oxygen, is a dull reddish purple color. A pulse oximeter calculates the ratio of these two forms of hemoglobin by measuring and comparing the transmission thorough skin of the pulsatile (and therefore arterial/arteriolar) components of two different colored light beams. The “oxygen saturation” is the percent of hemoglobin that is in its oxygenated form. Typically, healthy individuals have an oxygen saturation of 95%-100%. This is a useful parameter for monitoring patients who are undergoing surgery or who are sick, as a sudden drop in oxygen saturation may indicate that the lungs are not successfully oxygenating the blood. Then, corrective measures, such as providing the patient with supplemental oxygen may be undertaken. As useful as this information is, it does not indicate how well the body tissues are being oxygenated because oxygenation is a function of not just oxygen saturation, but also of tissue perfusion, that is, the amount of blood passing through the tissues. Tissue perfusion is not only important for oxygen delivery, but for multiple other functions, such as providing nutrients and carrying away waste products of metabolism from the tissues.

### SUMMARY OF THE INVENTION

A need thus exists in the art to indicate better how well the body tissues are being oxygenated.

It is thus an object of the invention to meet that need.

It is another object of the invention to measure both oxygen saturation and tissue perfusion.

To meet the above and other objects, tissue perfusion can be monitored with a new and different instrument called a pulse flowmeter. This device uses electrical impedance and signal processing to measure the small change in volume (“Pulse Volume”) of a limb segment, such as the forearm that occurs with each heartbeat. “Pulsatile Flow” is defined as the pulse volume multiplied by the heart rate and is one index of tissue perfusion. Pulsatile flow divided by limb segment volume is yet another index of tissue perfusion. The combination of knowing oxygen saturation and tissue perfusion is far more useful than knowing just one or the other of these parameters.

### BRIEF DESCRIPTION OF THE DRAWINGS

Preferred embodiments of the present invention will be set forth in detail with reference to the drawings, in which:

FIG. 1 shows pulse volume measurement;

FIG. 2 shows an electrode usable in the pulse volume measurement of FIG. 1;

FIG. 3 shows a modification of the electrode of FIG. 2 to include an oximetry sensor; and

FIG. 4 is a flow chart of selective signal averaging.

### DETAILED DESCRIPTION OF THE PREFERRED EMBODIMENTS

Preferred embodiments of the present invention will be set forth in detail with reference to the drawings, in which like reference numerals refer to like elements throughout.

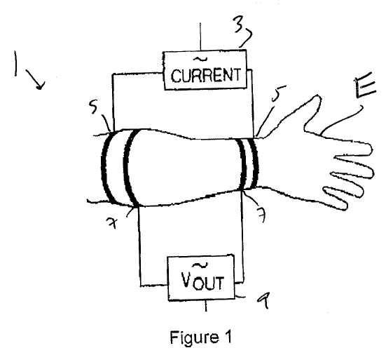

Limb segment impedance is measured with a quadripolar electrode arrangement shown in FIG. 1 as 1. An alternating electrical current (for example 1 milliamp, 40 kilohertz) is applied from a source 3 to the outer electrodes 5 attached to a patient's extremity (e.g., arm or leg) E. The voltage between the inner electrodes 7 is measured by a suitable measurement device 9. The amplitude of the voltage divided by the amplitude of the current is the electrical impedance of the limb segment.

When the heart beats and pushes blood through the limb segment, the cross section of the limb segment gets a little bigger, and its impedance decreases. This change in impedance can be used to calculate the pulse volume. The pulse volume may be expressed as an absolute number (ml), or may be normalized to limb segment length (ml/cm) or to limb segment volume (ml/cm³). Pulsatile flow may be expressed as an absolute number (ml/min), or may be normalized to limb segment length (ml/cm/min) or to limb segment volume (ml/min/cm³).

The most basic way to integrate an oximeter and a flowmeter is to physically combine them in one monitoring device. The two types of information could simply be displayed side-by-side in one instrument. However, there are more substantial ways to integrate these two types of instruments, so that “the whole is greater than the sum of its parts,” in other words, the combined instrument provides more benefits than just the two instruments side-by-side. Four ways to do this are described below. They include integration of the probe and electrode, software integration, display integration and calculation of new parameters. These can be used individually or in any suitable combination.

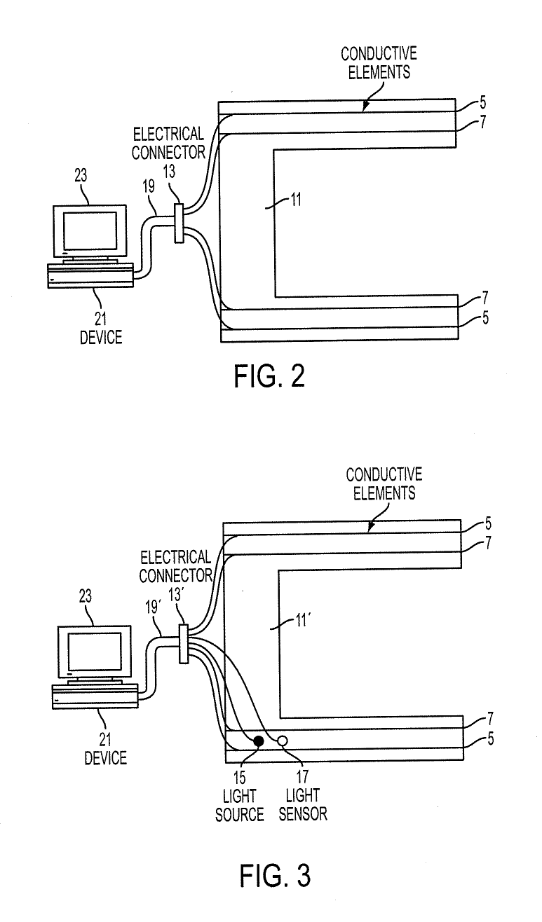

Integrated Probe-Electrode: One of the attractive features of a pulse oximeter is that the part of the device that contacts the patient, the “probe,” must only contain a light source, a light sensor and an attachment to hold them against the patient's skin. Typically the source and sensor are attached on opposite sides of a patient's finger. The light is transmitted through the finger. This is called a “Transmittance Pulse Oximeter.” Another arrangement, more suitable to a body part larger than a finger has the source and sensor more or less side-by-side. The light is reflected from the skin to the sensor. This is called a “Reflectance Pulse Oximeter.” The pulse flowmeter uses a non-invasive quadripolar circumferential electrode, such as that shown in FIG. 2 as 11, applied to an extremity (e.g., leg, arm, finger). The parts of the electrode containing the conductive elements are wrapped about the extremity and secured with hook-loop fastening material or any other suitable fastener. The pulse oximetry probe and pulse flowmeter electrode can be physically combined so that only one monitoring device need be attached to the patient and so that the wires connecting the probe/electrode to the device can be combined into a single cable, which can be attached to the electrode via an electrical connector 13. This connector can then be attached to a cable 19 which is connected to a computing device 21 and a display 23. The quadripolar electrode 11 is scalable to any size, so that can be applied to a variety of limb segments including a limb or a finger. In the case of a finger electrode, the optical source and sensor (in a typical transmittance pulse oximetry arrangement) can be integrated with the electrode; for example, as shown in FIG. 3, the optical source 15 and the sensor 17 are integrated into the electrode 11′, and the electrical connector 13′ includes contacts for the optical source 15 and the sensor 17; The quadripolar electrode can be scaled down to fit any size limb or finger segment. In the case of an arm or leg electrode, reflectance pulse oximetry would be used, as the light cannot penetrate through the full thickness of an arm or leg. In such an electrode, the light source 15 and sensor 17 would be used in a reflectance pulse oximetry arrangement and could be positioned on the vertical part of the electrode, i.e. the part of the electrode that is a bridge connecting the parts that wrap around the limb

The electrode of FIG. 2 or 3 can be connected by a suitable cable 19 or 19′ to a the above-noted computing device 21, which is programmed to implement the processing described herein and includes the above-noted display 23.

Display Integration—Modification of Pulse Oximetry Curve with Pulse Flow Data: The amplitude of the optical plethysmograph that constitutes the “pulse oximetry curve” does not measure tissue perfusion. Attempts have been made to get some perfusion information from this curve. One company computes the ratio of the pulsatile to nonpulsatile components of the infrared signal transmitted through the finger to calculate a “perfusion index.” This may provide some useful trend data but inherently can not actually measure tissue perfusion. In any of the preferred embodiments, the amplitude of the pulse oximetry curve can be adjusted in the device 21 to the actual measurement of pulse volume by the pulse flowmeter, making it more reflective of true tissue perfusion. The display could show either or both of the pulse oximetry and pulse volume curves, chosen either by the user or by an algorithm determining which is more suitable or of better quality. The display would show the pulse oximetry reading as well as the pulse flow reading.

Software Integration—Using the Pulse Oximetry Waveform to Provide a Gating Signal for Pulse Flowmeter: Pulse Flowmetry has traditionally used the QRS complex of the EKG waveform to provide a gating signal for a selective signal averaging process which is applied to the impedance waveforms. In the preferred embodiments, the pulse oximeter provides a pulsatile waveform, which occurs in a fixed time relationship to the cardiac cycle. The pulse oximeter waveform (or some waveform derived from the pulse oximeter waveform, such as its first derivative), can be used to provide an alternate gating signal for the selective signal averaging process. The device 21 can be programmed to perform the appropriate processing. More generally, an optical plethysmogram curve can be used to provide the gating signal. The optical plethysmogram curve can be processed identically to the EKG in the prior art to obtain the gating signal.

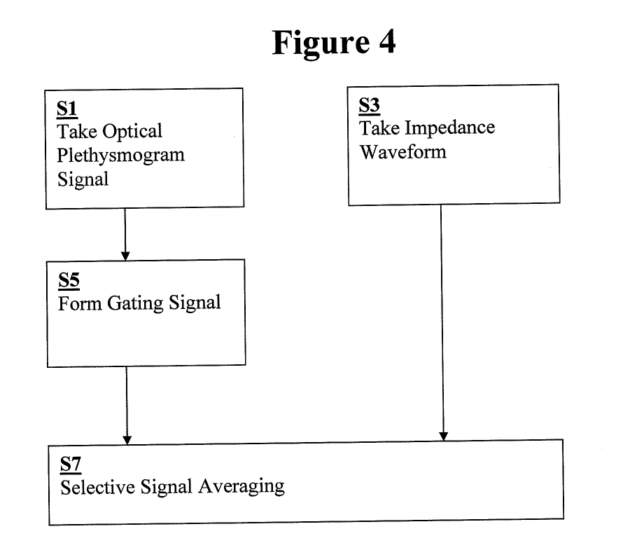

An overview is shown in the flow chart of FIG. 4. The optical plethysmogram curve and impedance waveform are taken in steps S1 and S3, respectively. The gating signal is formed in step S5 and used in the selective signal averaging of step S7.

This eliminates the need for the multiple, adhesive EKG electrodes By combining the instruments in this manner, not only are the extra wires of the EKG eliminated, but there also is no need for an adhesive attachment. This is particularly useful when a patient has a significant amount of hair in the area where the EKG electrodes are to be placed. This may introduce noise in the EKG waveform as well as result in discomfort for the patient when removing the electrodes. This type of integration provides “the best of both worlds” by keeping the attractive non-adhesive simple application of a pulse oximetry probe while providing quantitative assessment of peripheral perfusion. If the electrodes are also physically integrated (or in close proximity to one another), there needs to be only one cable between the patient and the monitoring device. If there is no need for pulse oximetry data, any optical plethysmogram can be used to provide the gating signal.

Sometimes pulse oximetry curves vary in quality. For example, a pulse oximetry probe placed on the finger of an arm that also has a blood pressure cuff on it will intermittently lose its signal. It may be desirable to have a plurality of pulse oximetry probes so that if a signal is lost in one, it will still be provided by another. These signals could be added, or processed with a suitable algorithm to insure that there is continuity of the optical plethysmogram for use as a gating signal.

Calculating new parameters. The oxygen saturation can be, for example, multiplied by the tissue perfusion to provide a new index that would better reflect tissue oxygenation than would pulse oximetry measurement alone. This new number could be displayed alone or in combination with the other parameters described above. It should be apparent that other mathematical combinations of these various indices could be derived.

While preferred embodiments of the invention have been set forth above, those skilled in the art who have reviewed the present invention will readily appreciate that other embodiments can be realized within the scope of the invention. For example, numerical values are illustrative rather than limiting. Also, both human and animal patients can be supported. Moreover, the optical plethysmogram does not have to be generated by a pulse oximeter; instead, it can be generated by simple optical plethysmograph. Therefore, the present invention should be construed as limited only by the appended claims.

We claim:

1. A system for providing oxygenation and perfusion data relating to bodily tissues in a patient, the system comprising:
an oximeter for measuring oxygen saturation in the patient's blood; and
an electrode for measuring a flow of the blood through the bodily tissues.

2. The system of claim 1, wherein the oximeter comprises a light source and a light sensor.

3. The system of claim 2, wherein the light source and the light sensor are integrated into the electrode.

4. The system of claim 3, wherein the electrode comprises an impedance electrode.

5. The system of claim 4, wherein the electrode comprises a quadripolar impedance electrode.

6. The system of claim 1, wherein the electrode comprises an impedance electrode.

7. The system of claim 6, wherein the electrode comprises a quadripolar impedance electrode.

8. The system of claim 1, wherein the processor is programmed to adjust an amplitude of a pulse oximetry curve to an measurement of pulse volume.

9. The system of claim 1, further comprising a processor which is programmed to process the measured saturation and flow.

10. The system of claim 9, wherein the processor is programmed to derive a gating signal from an optical plethysmogram curve and to use the gating signal in selective signal averaging of a pulsatile waveform.

11. The system of claim 10, wherein the optical plethysmogram curve is generated by the oximeter.

12. The system of claim 11, wherein the oximeter comprises a plurality of oximetry probes, and wherein the processor uses signals from the plurality of oximetry probes to derive the gating signal when one of the oximetry probes provides a bad signal.

13. The system of claim 12, wherein the processor derives the gating signal by selecting a good signal from another one of the oximetry probes.

14. The system of claim 12, wherein the processor derives the gating signal by adding the signals from the plurality of oximetry probes.

15. The system of claim 9, wherein the processor is programmed to derive an index of tissue oxygenation from the measured saturation and flow.

16. The system of claim 1, further comprising a display.

17. The system of claim 16, wherein the display is configured to display at least one of a pulse oximetry curve and a pulse volume curve.

18. A method for measuring oxygenation of bodily tissues in a patient, the method comprising:
(a) measuring oxygen saturation in the patient's blood, using an oximeter;
(b) measuring a flow of the blood through the bodily tissues, using an electrode; and
(c) providing information relating to the oxygenation in a processor in accordance with the oxygen saturation and the flow of the blood.

19. The method of claim 18, wherein the oximeter comprises a light source and a light sensor.

20. The method of claim 19 wherein the light source and the light sensor are integrated into the electrode.

21. The method of claim 20 wherein the electrode comprises an impedance electrode.

22. The method of claim 21, wherein the electrode comprises a quadripolar impedance electrode.

23. The method of claim 18, wherein the electrode comprises an impedance electrode.

24. The method of claim 23, wherein the electrode comprises a quadripolar impedance electrode.

25. The method of claim 18, wherein step (c) comprises adjusting an amplitude of a pulse oximetry curve to an measurement of pulse volume.

26. The method of claim 17, wherein step (c) comprises deriving a gating signal from an optical plethysmogram curve and to using the gating signal in selective signal averaging of a pulsatile waveform.

27. The method of claim 26, wherein the optical plethysmogram curve is generated by the oximeter.

28. The method of claim 27, wherein the oximeter comprises a plurality of oximetry probes, step (a) is performed using the plurality of oximetry probes, the gating signal is derived from signals from the plurality of oximetry probes when one of the oximetry probes provides a bad signal.

29. The method of claim 28, wherein the gating signal is derived by selecting a good signal from another one of the oximetry probes.

30. The method of claim 28, wherein the gating signal is derived by adding the signals from the plurality of oximetry probes.

31. The method of claim 18, wherein step (c) comprises deriving an index of tissue oxygenation from the measured saturation and flow.

32. The method of claim 18, further comprising displaying at least one of a pulse oximetry curve and a pulse volume curve.
